(19) United States
(12) Patent Application Publication
(10) Pub. No.: US 2015/0239839 A1
(43) Pub. Date: Aug. 27, 2015

(54) PROCESS FOR PHOTOCATALYTIC ACCEPTOR-FREE DEHYDROGENATION OF HYDROCARBAZOLES AND HYDROINDOLES

(71) Applicants: Robert FRANKE, Marl (DE); Jennifer JULIS, Duesseldorf (DE); Dirk FRIDAG, Haltern am See (DE); Katrin Marie DYBALLA, Recklinghausen (DE); Nico WEDING, Luedenscheid (DE); Abhishek Dutta CHOWDHURY, West Bengal (IN); Matthias BELLER, Nienhagen (DE); Ralf JACKSTELL, Cuxhaven Altenwalde (DE); Ivana FLEISCHER, Regensburg (DE); Beatrice PERCIA, Vadamadurai (IN)

(72) Inventors: Robert FRANKE, Marl (DE); Jennifer JULIS, Duesseldorf (DE); Dirk FRIDAG, Haltern am See (DE); Katrin Marie DYBALLA, Recklinghausen (DE); Nico WEDING, Luedenscheid (DE); Abhishek Dutta CHOWDHURY, West Bengal (IN); Matthias BELLER, Nienhagen (DE); Ralf JACKSTELL, Cuxhaven Altenwalde (DE); Ivana FLEISCHER, Regensburg (DE); Beatrice PERCIA, Vadamadurai (IN)

(73) Assignee: Evonik Industries AG, Essen (DE)

(21) Appl. No.: 14/629,095

(22) Filed: Feb. 23, 2015

(30) Foreign Application Priority Data
Feb. 25, 2014 (DE) 102014203345.4

(51) Int. Cl. C07D 209/86

(52) U.S. Cl. CPC C07D 209/86

(57) ABSTRACT

A process for the photocatalytic acceptor-free dehydrogenation of hydrocarbazoles and hydroindoles is provided, wherein a hydrocarbazole or hydroindole is irradiated in the presence of a catalyst that is a rhodium complex containing organic phosphorus(III) compounds as ligands.

### CROSS REFERENCE TO RELATED APPLICATIONS

This application claims priority to German Application No. 102014203345.4 filed Feb. 25, 2014, the disclosure of which is incorporated herein by reference in its entirety.

### BACKGROUND OF THE INVENTION

### Field of the Invention

The invention relates to a process for the photocatalytic acceptor-free dehydrogenation of hydrocarbazoles and hydroindoles.

Acceptor-free dehydrogenation is regarded as an ideal process because of its simplicity and atom economy. Such an atom-economic and acceptor-free dehydrogenation can be achieved under photocatalytic conditions. However, this reaction is limited by long reaction times, catalyst deactivation and lower reactivity compared to dehydrogenation/transfer hydrogenation reaction. Nomura et al. (J. Chem. Soc., Chem. Commun. 1988, 161-162) describes that the catalyst Rh(PMe₃)₂(CO)Cl is active in acceptor-free photocatalytic dehydrogenation and in thermochemical transfer hydrogenation, but only under hydrogen pressure, which restricts its usability for the synthesis of alkenes.

In order to achieve dehydrogenation at relatively low temperature under homogeneous conditions, sacrificial olefins (acceptors) are normally used in order to overcome the highly endothermic nature of the reaction. In general, efficient alkane transfer hydrogenation is conventionally achieved only with a large excess of a sacrificial olefin (up to a 20-fold excess), which restricts potential viable applicability. The industrially relevant turnover numbers (TON) in these methods are also normally limited to about 1000 at sensible reaction times. Turnover numbers (TON) >1000 are only achieved with long reaction times of several days. Even though there have continuously been various efforts to achieve homogeneous catalytic alkane dehydrogenations in the last 30 years, there is considerable need for significant improvements, specifically for acceptor-free atom-economic alkane dehydrogenation.

At present, successful dehydrogenations depend mainly on the behaviour of various specific demanding pincer ligands and the thermal stability thereof (including that of the metal complexes thereof). In order to overcome the high endothermicity of the alkane dehydrogenation, high reaction temperatures of up to 250° C. or long reaction times of up to 3 days are normally employed. Therefore, reactivity is determined strictly by the thermal stability of the catalyst. A further problem is that of inhibition of the reaction by the olefin which is present either as the sacrificial olefin or as the product, especially in the case of relatively long reaction times.

It was therefore an object of the invention to develop a process for acceptor-free dehydrogenation which avoids olefins as acceptors and permits an atom-economic dehydrogenation, the intention being to avoid long reaction times, catalyst deactivation and low reactivity.

### SUMMARY OF THE INVENTION

This and other objects have been achieved according to the present invention, the first embodiment of which includes a process for dehydrogenation of a hydrocarbazole or a hydroindole, comprising: irradiating the hydrocarbazole or hydroindole in a reaction mixture comprising a rhodium complex to remove hydrogen from the hydrocarbazole or hydroindole; wherein a hydrogen acceptor is not present, and the rhodium complex comprises a ligand of an organic phosphorus(III) compound.

In one variant of the first embodiment the dehydrogenation is conducted in the presence of a Lewis base.

In another variant the dehydrogenation is conducted in a glass reactor or a metal reactor comprising glass.

In a further variant the hydrogen is removed from the reaction mixture.

In another variant, the rhodium complex is of formula (I):

Rh(L¹)₂(CO)X (I)

wherein L¹ is P(C₁-C₅alkyl)₃, and X is chloride, bromide or acetate.

In a further variant, the rhodium complex is of formula (II):

Rh₂(L²)₂(CO)₂(X)₂ (II)

wherein L² is P(Ph)₂(CH₂)ₙP(Ph)₂, n is from 1 to 3, and X is chloride, bromide or acetate.

In special embodiments the hydrocarbazole is dodecahydro-N-ethylcarbazole or dodecahydro-N-methylcarbazole.

The forgoing description is intended to provide a general introduction and summary of the present invention and is not intended to be limiting in its disclosure unless otherwise explicitly stated. The presently preferred embodiments, together with further advantages, will be best understood by reference to the following detailed description.

### DETAILED DESCRIPTION OF THE PREFERRED EMBODIMENTS

As used herein, the words “a” and “an” and the like carry the meaning of “one or more.” The phrases “selected from the group consisting of,” “chosen from,” and the like include mixtures of the specified materials. Terms such as “contain(s)” and the like are open terms meaning ‘including at least’ unless otherwise specifically noted. Where a numerical limit or range is stated, the endpoints are included. Also, all values and subranges within a numerical limit or range are specifically included as if explicitly written out.

The invention provides a process for the photocatalytic acceptor-free dehydrogenation of hydrocarbazoles and hydroindoles, characterized in that a hydrocarbazole or hydroindole is irradiated in the presence of a rhodium complex containing organic phosphorus(III) compounds as ligands as catalyst.

Thus, in the first embodiment the present invention provides a process for dehydrogenation of a hydrocarbazole or a hydroindole, comprising:

irradiating the hydrocarbazole or hydroindole in a reaction mixture comprising a rhodium complex to remove hydrogen from the hydrocarbazole or hydroindole;

wherein a hydrogen acceptor is not present, and

the rhodium complex comprises a ligand of an organic phosphorus(III) compound.

In a variant of the process, the hydrocarbazole is dodecahydro-N-ethylcarbazole or dodecahydro-N-methylcarbazole.

In one variant of the process, the dehydrogenation is carried out with addition of a Lewis base. In one variant of the process, the Lewis base is an organic amine.

In one variant of the process, the Lewis base is a heterocyclic amine.

In one variant of the process, the Lewis base is a bipyridine.

In one variant of the process, the Lewis base used is 2,2-bipyridine or 4,4-bipyridine.

In one variant of the process, the Lewis base used is bathocuproin or phenanthroline.

In one variant of the process, the reaction is carried out in the presence of CO₂.

It has additionally been found that the dehydrogenation reactions proceed better with higher yields in a longer reaction regime in the presence of CO₂.

In one variant of the process, irradiation is effected with light having a wavelength of from 320 nm to 500 nm.

The influence of the wavelength of the light source used has also been studied, and it was found that irradiation is preferably effected by light having a wavelength of λ=320 nm to 500 nm.

In one variant of the process, the dehydrogenation is carried out at a temperature of from 45° C. to 120° C.

The inventive dehydrogenation also takes place in a particularly optimal manner at a temperature of preferably from 45° C. to 120° C. A temperature of from 80° C. to 95° C. may be particularly preferred. The best yields may be achieved at a reaction temperature of from 85° C. to 90° C.

In one variant of the process, a rhodium complex of formula (I) is used:

Rh(L¹)₂(CO)X (I),

wherein

L¹ is P(C₁-C₅alkyl)₃,

and X is an anion selected from chloride, bromide, and acetate.

In one variant of the process, L¹ is PMe3 or PtBu₃.

In one variant of the process, X is chloride.

In one variant of the process, a rhodium complex of formula (II) is used:

Rh₂(L²)₂(CO)₂(X)₂ (II),

wherein

L² is P(Ph)₂(CH₂)ₙP(Ph)₂,

n is 1 to 3, and X is chloride, bromide or acetate.

In one variant of the process, L² is P(Ph)₂(CH₂)P(Ph)₂.

In one variant of the process, X is chloride.

In one variant of the process, the hydrogen formed may be removed from the reaction mixture.

In order to conduct an even more effective reaction, the hydrogen formed can advantageously be removed from the reaction solution. This is achieved, for example, by application of a high stirrer speed and/or a constant gas stream (e.g. argon). This reaction regime may further increase the TONs achieved, and they are thus significantly higher than in systems previously described.

In one variant of the process, irradiation may be effected through a glass plate.

Furthermore, the use of a glass reactor or of a metal reactor including glass in the process according to the invention may be particularly effective. Single-wall glass reactors, preferably having a wall thickness of from 1.0 to 3.0 mm, may be particularly suitable. Interestingly, barely any reaction takes place if merely a suitable metal reactor with a light inlet is used. Surprisingly, the presence of glass has a remarkable influence on the inventive reaction, and not only the material but also the wall thickness of the glass vessel used may be important. Thus, the reaction may be conducted using glass vessels of various wall thicknesses; a wall thickness of from 1.2 mm to 1.8 mm has been found to be particularly optimal.

In one variant of the process, the glass plate has a thickness of from 1.2 mm to 3.0 mm.

Advantageously, therefore, thin-walled glass vessels may be used in the process according to the invention, since transmission therein is considerably greater and therefore more energy is available for dehydrogenation. This significant influence of the presence of glass and of the wall thickness of the reaction vessels has not been previously reported in photocatalytic reactions.

In one variant of the process, a single-walled glass reactor may be used.

The irradiation may preferably be effected by light, preferably having a wavelength range of λ=320 nm to 500 nm. The dehydrogenation may also be effected in a particularly optimal manner at a temperature of preferably 45° C. to 120° C., preferably at a temperature of 80° C. to 95° C. In particular, a reaction temperature of from 85° C. to 90° C. may be effective. In addition, the use of a glass reactor or of a metal reactor including glass in the process according to the invention is particularly effective. Single-walled glass reactors, preferably having a glass thickness of from 1.0 to 3.0 mm, may be particularly suitable. The rhodium complex used may preferably be one of formula (I), as specified above, especially with L¹=PMe₃ or PBu₃ and X=Cl. In general, the preferred catalyst may be Rh(PMe₃)₂(CO)Cl.

The process can be used for the photocatalytic acceptor-free dehydrogenation of hydrocarbazoles, in which a hydrocarbazole is irradiated in the presence of a rhodium complex containing organic phosphorus(III) compounds as ligands as catalyst. The hydrocarbazole may preferably be dodecahydro-N-ethylcarbazole or dodecahydro-N-methylcarbazole.

In another variant according to the invention, the process can also be used for the photocatalytic acceptor-free dehydrogenation of a hydroindoline, in which the hydroindoline is irradiated in the presence of a rhodium complex containing organic phosphorus(III) compounds as ligands as catalyst. The hydroindoline may preferably be indoline.

The dehydrogenation of hydrocarbazoles such as dodecahydro-N-ethylcarbazole may be effectively realized photocatalytically under solvent-free conditions. It has been reported that complete hydrogenation of N-ethylcarbazole to dodecahydro-N-ethylcarbazole as chemical hydrogen store is achieved after two days using 5% Ru/Al₂O₃. [Morawa Eblagon, K.; Tam, K.; Kerry Yu, K. M.; Zhao, S.-L.; Gong, X.-Q.; He, H.; Ye, L.; Wang, L.-C.; Ramirez-Cuesta, A. J.; Tsang, S. C. J. Phys. Chem. C 2010, 114, 9720-9730]. As reported, the desired dehydrogenation occurs only at very high temperatures of 200° C. in the presence of heterogeneous catalysts, while in the photocatalytic process of the invention both fully and partially dehydrogenated carbazoles may be obtained at preferred temperatures of about 90° C.

Thus, the process represents a suitable homogeneously catalyzed method for mild dehydrogenation of potential chemical hydrogen stores.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

The invention is illustrated in detail hereinafter by working examples.

### EXAMPLES

### General Reaction Conditions

All synthetic operations were conducted under argon in dried Duran borosilicate glass vessels using suitable Schlenk techniques. The Rh(PMe₃)₂(CO)Cl catalyst was prepared in a manner analogous to a literature method. [Bridgewater, J. S.; Netzel, T. L.; Schoonover, J. R.; Massick, S. M.; Ford, P. C. Inorg. Chem. 2001, 40, 1466-1476]. The products were analysed in comparison with a comparative sample and the yield by means of gas chromatography (Agilent 6890N network GC System using a (60 m×250 μm×0.25 μm) DB Wax column and isooctane as internal standard (0.2 ml, 1.2 mmol) after dilution with acetone. The response factor of each product was determined by a Multiple Point Internal Standard GC Quantification Method′ against isooctane. For all analyses, the following conditions were chosen: N₂ as carrier gas, inlet temperature: 250° C., inlet pressure: 104.4 kPa, injection volume: 1.0 μl, split ratio: 100:1, split flow: 80.0 ml/min, flow rate: 0.8 ml/min up to 20 min, and was then increased to 2.8 ml/min at 1.0 ml/min², temperature: 35° C. to 20 min, then increased at 40° C./min to 200° C. and then held at 200° C. for 15 min. Detector temperature: 250° C., hydrogen flow rate: 30 ml/min, air: 300 ml/min, nitrogen flow rate: 25 ml/min.

General procedure 1: The appropriate glass vessel, provided with a reflux condenser and magnetic stirrer, was charged under argon with 0.004 mmol of the Rh(PMe₃)₂(CO)Cl catalyst and 0.02 mmol of the appropriate additive. Very careful working under argon as inert gas was necessary, since the catalyst is deactivated very readily in the presence of atmospheric oxygen and light. Subsequently, 30 mmol of substrate was added and an argon stream applied. The stirrer speed was set to 1000 min⁻¹ and the glass vessel was covered with aluminium foil. The Lumatec Superlite 400 light source used, which emits light over a wavelength range from 320 nm to 500 nm, was then switched on. After the reaction, the light source was switched off, the reaction solution was cooled down and the yield was determined by gas chromatography using isooctane as internal standard. The turnover numbers (TON) in the tables are calculated as [mmol of product]/[mmol of catalyst].

General procedure 2: The appropriate glass vessel, provided with a reflux condenser and magnetic stirrer, was charged under argon with 0.004 mmol of the Rh(PMe₃)₂(CO)Cl catalyst and 0.02 mmol of the appropriate additive. Very careful working under argon as inert gas was necessary, since the catalyst is deactivated very readily in the presence of atmospheric oxygen and light. Subsequently, 30 mmol of substrate was added and an argon stream was applied. The stirrer speed was set to 1000 min⁻¹ and the glass vessel was covered with aluminium foil. A metal capillary was used to pass a CO₂ stream through the solution. The Lumatec Superlite 400 light source used was then switched on. After the reaction, the light source was switched off, the CO2 stream was stopped, the reaction solution was cooled down and the yield was determined by gas chromatography using isooctane as internal standard. The turnover numbers (TON) in the tables are calculated as [mmol of product]/[mmol of catalyst].

### EXAMPLE

### Dehydrogenation of dodecahydro-N-ethylcarbazole (H₁₂NEC)

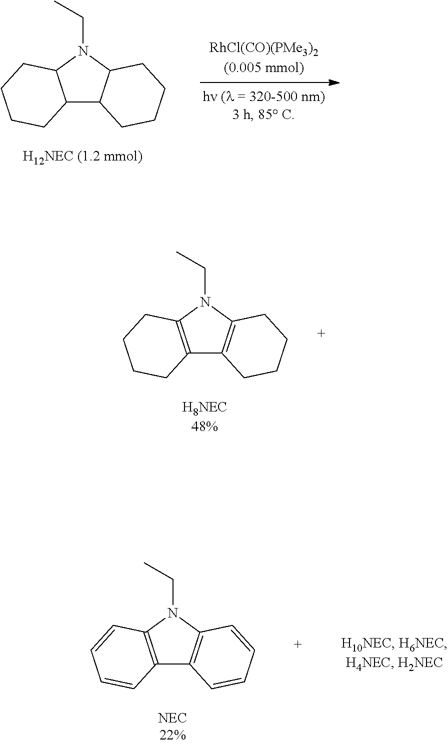

A Schlenk vessel having a wall thickness of 1.2 mm, provided with a reflux condenser and magnetic stirrer, was charged with 1.6 mg of Rh(PMe₃)₂(CO)Cl (0.005 mmol). Subsequently, the reaction vessel was evacuated and filled with argon three times, in order to achieve inert conditions. Then 250 ml of dodecahydro-N-ethylcarbazole (1.2 mmol) were added. An argon stream was applied in order to remove hydrogen formed. The reactor was covered with aluminium foil and the light source having a wavelength of 320-500 nm (Lumatec Superlite 400) was switched on. The mixture was stirred at 1000 min⁻¹ for three hours. After the reaction, the light source was switched off and the reaction solution is cooled down. The yield was determined by gas chromatography using isooctane as internal standard.

TABLE

| Substrate | Time (h) | Yield (%) | TON | TOF (h⁻¹) |
|---|---|---|---|---|
| H₁₂NEC | 3 | 80 | 188 | 63 |

As can be seen from the table, the product can be obtained in a very good yield within 3 hours.

1. A process for dehydrogenation of a hydrocarbazole or a hydroindole, comprising:
irradiating the hydrocarbazole or hydroindole in a reaction mixture comprising a rhodium complex to remove hydrogen from the hydrocarbazole or hydroindole;
wherein
a hydrogen acceptor is not present, and
the rhodium complex comprises a ligand of an organic phosphorus(III) compound.

2. The process according to claim 1,
wherein a hydrocarbazole is dehydrogenated, and
the hydrocarbazole is at least one of dodecahydro-N-ethylcarbazole and dodecahydro-N-methylcarbazole.

3. The process according to claim 1,
wherein the reaction mixture further comprises a Lewis base.

4. The process according to claim 3,
wherein the Lewis base is an organic amine.

5. The process according to claim 3,
wherein the Lewis base is a heterocyclic amine.

6. The process according to claim 3,
wherein the Lewis base is at least one selected from the group consisting of a bipyridine, bathocuproin and phenanthroline.

7. The process according to claim 6,
wherein the Lewis base is a bipyridine and is 2,2-bipyridine and/or 4,4-bipyridine.

8. The process according to claim 6,
wherein the Lewis base is bathocuproin or phenanthroline.

9. The process according to claim 1,
wherein the reaction is conducted in the presence of CO₂.

10. The process according to claim 1,
wherein the irradiation is conducted with light of wavelength 320 nm to 500 nm.

11. The process according to claim 1,
wherein a temperature of the dehydrogenation is from 45° C. to 120° C.

12. The process according to claim 1,
wherein the rhodium complex is of formula (I):
Rh(L¹)₂(CO)X (I)
wherein
L¹ is P(C₁-C₅alkyl)₃, and
X is chloride, bromide or acetate.

13. The process according to claim 12, wherein
L¹ is PMe₃ or PtBu₃.

14. The process according to claim 12, wherein X is chloride.

15. The process according to claim 1, wherein the rhodium complex is of formula (II):
Rh₂(L²)₂(CO)₂(X)₂ (II)
wherein
L² is P(Ph)₂(CH₂)ₙP(Ph)₂,
n is from 1 to 3, and
X is chloride, bromide or acetate.

16. The process according to claim 15, wherein
L² is P(Ph)₂(CH₂)P(Ph)₂.

17. The process according to claim 15, wherein X is chloride.

18. The process according to claim 1, further comprising removing the hydrogen from the reaction mixture.

19. The process according to claim 1, wherein the dehydrogenation is conducted in a glass reactor or a metal reactor comprising glass.

20. The process according to claim 1, wherein the dehydrogenation is conducted in a single-wall glass reactor having a wall thickness of from 1.0 to 3.0 mm.
